(19) United States
(12) Patent Application Publication
(10) Pub. No.: US 2025/0160775 A1
(43) Pub. Date: May 22, 2025

(54) RADIOGRAPHY SYSTEM, CONTROL METHOD, AND CONTROL PROGRAM

(71) Applicant: FUJIFILM CORPORATION, Tokyo (JP)

(72) Inventors: Hisatsugu HORIUCHI, Kanagawa (JP); Koji TANINAI, Kanagawa (JP); Tomoaki NAKASHIMA, Kanagawa (JP); Kyohei MORI, Kanagawa (JP); Naoyuki NISHINO, Kanagawa (JP)

(21) Appl. No.: 18/942,796

(22) Filed: Nov. 11, 2024

(30) Foreign Application Priority Data
Nov. 20, 2023 (JP) 2023-196965

(51) Int. Cl. A61B 6/00; A61B 6/46

(52) U.S. Cl. CPC A61B 6/4452; A61B 6/4411; A61B 6/461; A61B 6/547

(57) ABSTRACT

A radiography system includes: a radiation source unit; a panel unit; and a processor. The radiation source unit includes, a radiation emitting unit, a radiation source base portion that supports the radiation emitting unit and includes a radiation source traveling mechanism for performing a traveling movement, and a first detection unit provided at the radiation source base portion. The panel unit includes, a panel that detects radiation, a panel base portion that supports the panel and includes a panel traveling mechanism for performing a traveling movement, and a second detection unit provided at the panel base portion. The radiation source base portion and the panel base portion have a common shape. The processor controls a relative position or posture of the radiation source unit and the panel unit on the basis detection results of the first detection unit or the second detection unit.

### CROSS-REFERENCE TO RELATED APPLICATIONS

This application claims priority from Japanese Application No. 2023-196965, filed on Nov. 20, 2023, the entire disclosure of which is incorporated herein by reference.

### BACKGROUND

### Technical Field

The present disclosure relates to a radiography system, a control method, and a control program.

### Related Art

In the capturing of radiation images, a radiation source and a panel that detects radiation are disposed to face each other. As a technique for this, for example, JP2000-166906A discloses a technique in which positions of robot arms of both robots, which are a robot in which a radiation source as an X-ray source is disposed on a robot arm and a robot in which an X-ray receiver as a panel that detects radiation is disposed on a robot arm, are controlled such that the X-ray source faces the X-ray receiver.

Meanwhile, there is a radiography system in which a radiation source unit including a radiation source and a panel unit including a panel that detects radiation are separate bodies and can travel individually. In such a manner, in a case where the radiation source unit and the panel unit can travel individually, it may be difficult to perform alignment of the radiation source and the panel.

### SUMMARY

The present disclosure has been made in consideration of the circumstances described above, and an object of the present disclosure is to provide a radiography system, a control method, and a control program capable of easily perform alignment of a radiation source unit and a panel unit.

In order to achieve the above-described object, a radiography system according to a first aspect of the present disclosure comprises a radiation source unit; a panel unit; and at least one processor, in which the radiation source unit includes a radiation emitting unit that includes a radiation source, a radiation source base portion that supports the radiation emitting unit and includes a radiation source traveling mechanism for performing a traveling movement, and a first detection unit that is provided in the radiation source base portion, the panel unit includes a panel that detects radiation emitted from the radiation emitting unit, a panel base portion that supports the panel and includes a panel traveling mechanism for performing a traveling movement, and a second detection unit that is provided in the panel base portion, the radiation source base portion and the panel base portion have a common shape, and the processor further includes a control device that controls at least one of a relative position or a relative posture of the radiation source unit and the panel unit on the basis of at least one of a detection result of the first detection unit or a detection result of the second detection unit.

In a radiography system according to a second aspect of the present disclosure, in the radiography system according to the first aspect, the first detection unit and the second detection unit include an optical camera, the detection result of the first detection unit is a first optical image captured by the optical camera of the first detection unit, and the detection result of the second detection unit is a second optical image captured by the optical camera of the second detection unit.

In a radiography system according to a third aspect of the present disclosure, in the radiography system according to the second aspect, each of a housing of the radiation source base portion and a housing of the panel base portion is provided with a marker, and the control device controls at least one of the relative position or the relative posture of the radiation source unit and the panel unit on the basis of at least one of an image of the marker provided on the panel base portion included in the first optical image or an image of the marker provided on the radiation source base portion included in the second optical image.

In a radiography system according to a fourth aspect of the present disclosure, in the radiography system according to the third aspect, in a case where it is determined that a state of the image of the marker provided on the panel base portion included in the first optical image and a state of the image of the marker provided on the radiation source base portion included in the second optical image are the same, the control device determines that the radiation source unit and the panel unit are facing each other.

In a radiography system according to a fifth aspect of the present disclosure, in the radiography system according to the second aspect, each of a housing of the radiation source base portion and a housing of the panel base portion is provided with a plurality of markers including a predetermined marker, and the control device controls at least one of the relative position or the relative posture of the radiation source unit and the panel unit on the basis of an optical image in which an image of the predetermined marker is included, among the first optical image and the second optical image.

In a radiography system according to a sixth aspect, in the radiography system according to the first aspect, the control device derives a movement amount and a movement direction of at least one of the radiation source unit or the panel unit for making positions of the radiation source unit and the panel unit into at least one of a relative position or a relative posture determined in advance, on the basis of the detection result of at least one of the first detection unit or the second detection unit.

In a radiography system according to a seventh aspect, in the radiography system according to the sixth aspect, in a case where the movement amount and the movement direction of the radiation source unit are derived, the control device causes the radiation source unit to perform a traveling movement using the radiation source traveling mechanism, on the basis of the derived movement amount and movement direction, and in a case where the movement amount and the movement direction of the panel unit are derived, the control device causes the panel unit to perform a traveling movement using the panel traveling mechanism, on the basis of the derived movement amount and movement direction.

In a radiography system according to an eighth aspect, in the radiography system according to the sixth aspect, the control device displays the derived movement amount and movement direction on a display unit.

In order to achieve the above-described object, a control method according to a ninth aspect is a control method of a radiography system including a radiation source unit and a panel unit, in which the radiation source unit includes a radiation emitting unit that includes a radiation source, a radiation source base portion that supports the radiation emitting unit and includes a radiation source traveling mechanism for performing a traveling movement, and a first detection unit that is provided in the radiation source base portion, the panel unit includes a panel that detects radiation emitted from the radiation emitting unit, a panel base portion that supports the panel and includes a panel traveling mechanism for performing a traveling movement, and a second detection unit that is provided in the panel base portion, and the radiation source base portion and the panel base portion have a common shape, the control method comprising, via a processor, controlling at least one of a relative position or a relative posture of the radiation source unit and the panel unit on the basis of at least one of a detection result of the first detection unit or a detection result of the second detection unit.

In order to achieve the above-described object, a control program according to a tenth aspect is a control program of causing a processor of a control device that controls a radiography system including a radiation source unit and a panel unit, in which the radiation source unit includes a radiation emitting unit that includes a radiation source, a radiation source base portion that supports the radiation emitting unit and includes a radiation source traveling mechanism for performing a traveling movement, and a first detection unit that is provided in the radiation source base portion, the panel unit includes a panel that detects radiation emitted from the radiation emitting unit, a panel base portion that supports the panel and includes a panel traveling mechanism for performing a traveling movement, and a second detection unit that is provided in the panel base portion, and the radiation source base portion and the panel base portion have a common shape, to execute processing of controlling at least one of a relative position or a relative posture of the radiation source unit and the panel unit on the basis of at least one of a detection result of the first detection unit or a detection result of the second detection unit.

According to the present disclosure, it is possible to easily perform the alignment of the radiation source unit and the panel unit.

### BRIEF DESCRIPTION OF THE DRAWINGS

FIG. 1 is a configuration diagram schematically illustrating an example of an overall configuration of a radiography system according to an embodiment.

FIG. 2 is a side view illustrating an example of an external appearance of a radiation source unit of an embodiment.

FIG. 3 is a block diagram illustrating an example of a configuration of a radiation source unit of an embodiment.

FIG. 4 is a side view illustrating an example of an external appearance of a panel unit of an embodiment.

FIG. 5 is a block diagram illustrating an example of a configuration of a panel unit of an embodiment.

FIG. 6A is a diagram for describing a relationship between an image of a marker included in an optical image and positions of a radiation source unit and a panel unit.

FIG. 6B is a diagram for describing a relationship between an image of a marker included in an optical image and positions of a radiation source unit and a panel unit.

FIG. 7 is a functional block diagram of a radiation source unit and a panel unit of an embodiment.

FIG. 8 is a flowchart of an example of position control processing executed by a radiation source unit of an embodiment.

FIG. 9 is a flowchart of an example of position control processing executed by a panel unit of an embodiment.

FIG. 10 is a flowchart of another example of position control processing executed by a radiation source unit of an embodiment.

FIG. 11 is a flowchart of another example of the position control processing executed by a panel unit of an embodiment.

FIG. 12 is an example for describing a modification example of an embodiment.

FIG. 13 is a flowchart of another example of position control processing executed by a radiation source unit of an embodiment.

FIG. 14 is a flowchart of another example of position control processing executed by a panel unit of an embodiment.

FIG. 15 is a flowchart of an example of position checking processing.

### DETAILED DESCRIPTION

Hereinafter, an embodiment of the present invention will be described in detail with reference to the drawings. Note that the present embodiment does not limit the present invention.

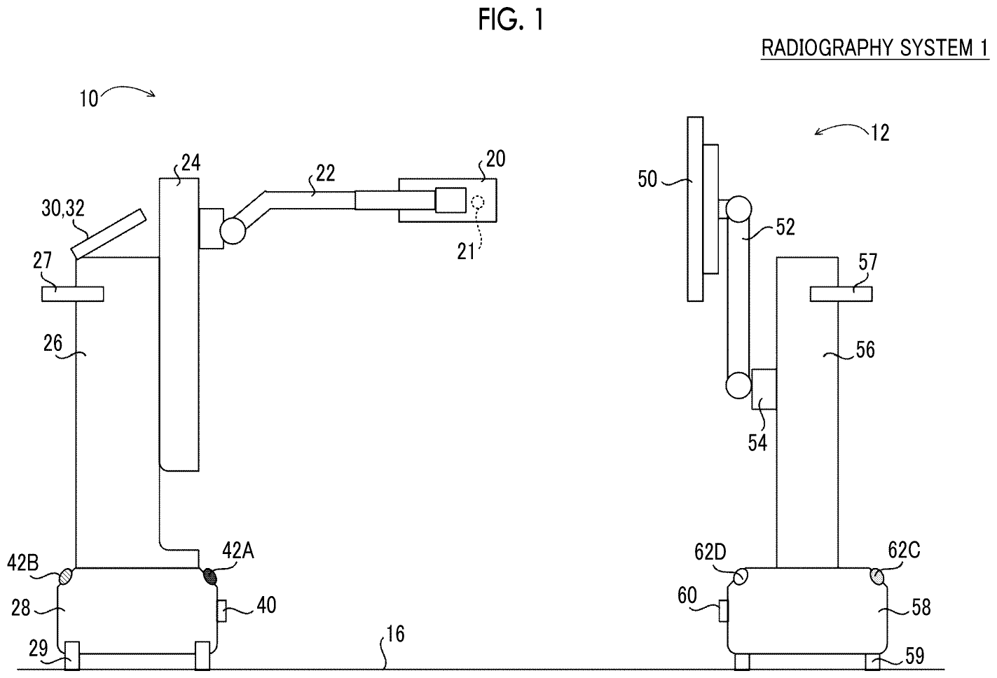

First, an example of an overall configuration of a radiography system of the present embodiment will be described. FIG. 1 is a configuration diagram illustrating an example of an overall configuration of a radiography system 1 of the present embodiment.

As illustrated in FIG. 1, the radiography system of the present embodiment comprises a radiation source unit 10 and a panel unit 12.

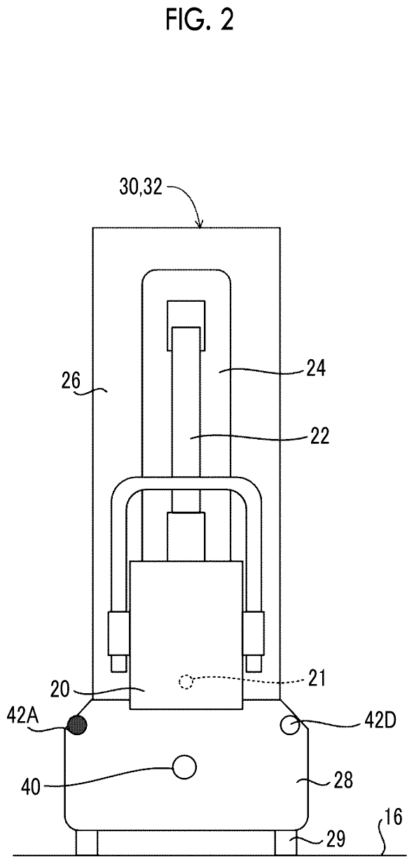

First, the configuration of the radiation source unit 10 will be described with reference to FIG. 2. Note that FIG. 1 illustrates a schematic diagram of an external appearance of the radiation source unit 10 as viewed from a side surface, and FIG. 2 illustrates a schematic diagram of the external appearance of the radiation source unit 10 as viewed from the front.

As illustrated in FIGS. 1 and 2, the radiation source unit 10 includes a radiation emitting unit 20 including a radiation source 21, a radiation source base portion 28 that supports the radiation emitting unit 20 and includes four wheels 29 for performing a traveling movement, and an optical camera 40 provided in the radiation source base portion 28.

The radiation emitting unit 20 including the radiation source 21 is provided on an arm 22. The arm 22 is held by a holding part 24. A support portion 26 supports the radiation emitting unit 20 by supporting the holding part 24. The holding part 24 can be moved in an up-down direction, in other words, in a direction away from and in a direction approaching the radiation source base portion 28. An end portion of the arm 22, which is on a side opposite to the end portion where the radiation emitting unit 20 is provided, is connected to a shaft provided in the holding part 24, and the radiation emitting unit 20 can be moved in a direction to be spaced from the holding part 24 by rotating the arm 22 by the shaft. In the radiation source unit 10, the holding part 24 is moved in the up-down direction and the arm 22 is rotated, so that the position of the radiation source 21 of the radiation emitting unit 20 in a height direction can be adjusted.

In addition, an operation unit 30 and a display unit 32, which will be described in detail later, are provided on an upper surface of the support portion 26 so as to be accommodated. Furthermore, a gripping portion 27 to be gripped in a case where a user such as a technician moves the radiation source unit 10 is provided in the support portion 26. The support portion 26 is supported by the radiation source base portion 28.

The four wheels 29 are provided at four corners of the radiation source base portion 28, and traveling on a floor surface 16 or the like can be performed by rotating the four wheels 29. The wheel 29 of the present embodiment is an example of a radiation source traveling mechanism of the present disclosure. In addition, the optical camera 40 is provided on a front surface of a housing of the radiation source base portion 28. The optical camera 40 can image a panel base portion 58 of the panel unit 12 in a state of facing at least the radiation source unit 10. In addition, markers 42A to 42D are provided at four corners of the housing of the radiation source base portion 28. Note that, among the markers 42A to 42D, the markers 42C and 42D are not illustrated in FIG. 1, and the markers 42B and 42C are not illustrated in FIG. 2 since the markers are in the shadow of the radiation source base portion 28. The marker 42A to the marker 42D are markers that can be respectively identified, and the identification of the marker 42A to the marker 42D can be performed in at least an optical image captured by an optical camera 60 of the panel unit 12. For example, at least one of the shape or the color of each of the marker 42A to the marker 42D is different from the others. Note that, in the following, in a case where the marker 42A to the marker 42D are collectively referred to, the marker 42A to the marker 42D are simply referred to as the “marker 42”. The optical camera 40 of the present embodiment is an example of a first detection unit according to the present disclosure.

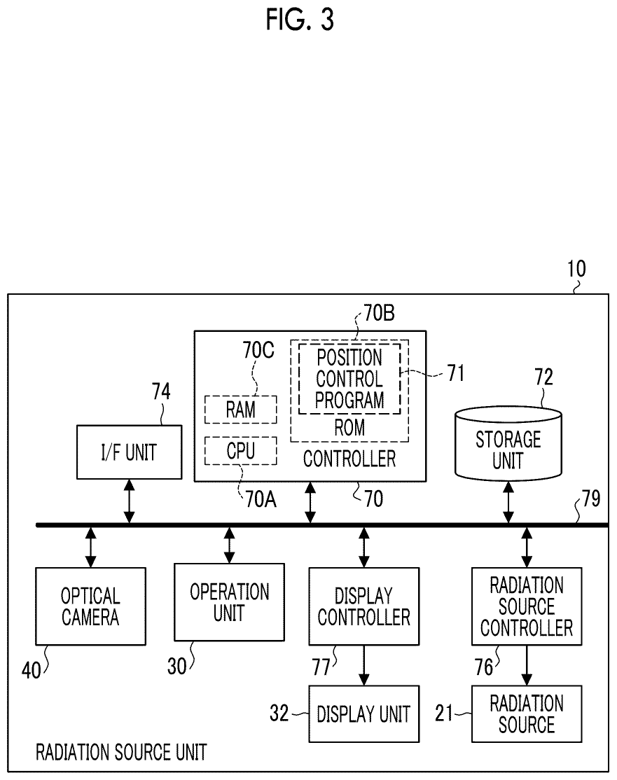

Furthermore, the configuration of the radiation source unit 10 will be described with reference to FIG. 3. As illustrated in FIG. 3, the radiation source unit 10 of the present embodiment further comprises a controller 70, a storage unit 72, a radiation source controller 76, and a display controller 77. The operation unit 30, the display unit 32, the optical camera 40, the controller 70, the storage unit 72, the radiation source controller 76, and the display controller 77 are connected to each other via a bus 79, such as a system bus or a control bus, such that various kinds of information can be exchanged.

The controller 70 includes a central processing unit (CPU) 70A, a read only memory (ROM) 70B, and a random access memory (RAM) 70C. The CPU 70A controls the entire radiation source unit 10. The ROM 70B stores in advance various programs including a position control program 71 to be executed by the CPU 70A. The RAM 70C temporarily stores various kinds of data.

The storage unit 72 stores various kinds of information related to imaging. The storage unit 72 is realized by, for example, a storage medium such as a hard disk drive (HDD), a solid state drive (SSD), and a flash memory.

An I/F unit 74 performs communication of various kinds of information with the panel unit 12 or an external apparatus by wired communication or wireless communication.

The operation unit 30 is used for a user to input an instruction related to imaging, various kinds of information, and the like. Note that the operation unit 30 is not particularly limited, and examples of the operation unit 30 include various switches, a touch panel, a touch pen, a mouse, and the like. The display unit 32 displays various kinds of information under the control of the display controller 77. Note that, in the present embodiment, a touch panel display in which the operation unit 30 and the display unit 32 are integrated is used as an example.

The radiation source controller 76 controls the radiation source 21 of the radiation emitting unit 20 on the basis of the control of the controller 70. The radiation source 21 emits radiation under the control of the controller 70.

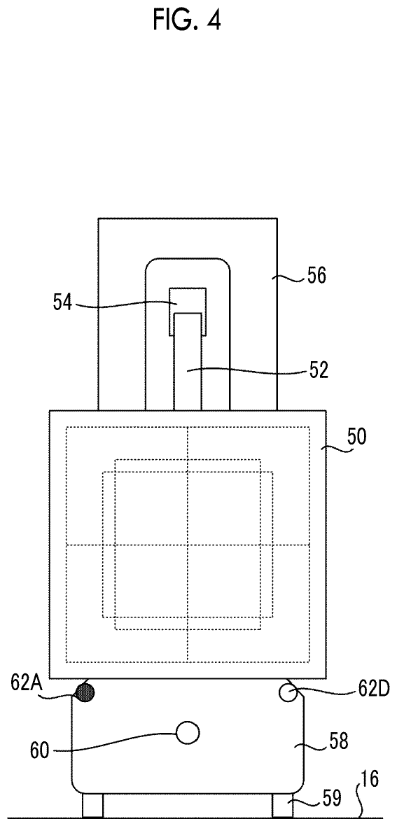

Next, the configuration of the panel unit 12 will be described with reference to FIG. 4. Note that FIG. 1 illustrates a schematic diagram of an external appearance of the panel unit 12 as viewed from a side surface, and FIG. 4 illustrates a schematic diagram of the external appearance of the panel unit 12 as viewed from the front.

As illustrated in FIGS. 1 and 4, the panel unit 12 includes a panel 50 that detects the radiation emitted from the radiation emitting unit 20, the panel base portion 58 that supports the panel 50 and includes four wheels 59 for performing a traveling movement, and the optical camera 60 that is provided in the panel base portion 58.

The panel 50 is provided on an arm 52. The arm 52 is held by a holding part 54. A support portion 56 supports the panel 50 by supporting the holding part 54. The holding part 54 can be moved in the up-down direction, in other words, in a direction away from and in a direction approaching the panel base portion 58. An end portion of the arm 52, which is on a side opposite to the end portion where the panel 50 is provided, is connected to a shaft provided in the holding part 54, and the panel 50 can be moved in a direction to be spaced from the holding part 54 by rotating the arm 52 by the shaft. In the panel unit 12, the holding part 54 is moved in the up-down direction and the arm 52 is rotated, so that the position of the panel 50 in the height direction can be adjusted.

In addition, a gripping portion 57 to be gripped in a case where a user such as a technician moves the panel unit 12 is provided in the support portion 56. The support portion 56 is supported by the panel base portion 58.

The panel base portion 58 and the radiation source base portion 28 have a common shape. Specifically, a region occupied by the panel base portion 58 in a case where the panel base portion 58 is projected onto the floor surface 16 and a region occupied by the radiation source base portion 28 in a case where the radiation source base portion 28 is projected onto the floor surface 16 have the same shape. In addition, in the present embodiment, the housing of the panel base portion 58 and the housing of the radiation source base portion 28 have the same shape except for the upper surface.

The four wheels 59 are provided at four corners of the panel base portion 58, and traveling on the floor surface 16 or the like can be performed by rotating the four wheels 59. The wheel 59 of the present embodiment is an example of a panel traveling mechanism of the present disclosure. In addition, the optical camera 60 is provided on a front surface of the housing of the panel base portion 58. The optical camera 60 can image the radiation source base portion 28 of the radiation source unit 10 in a state of facing at least the panel unit 12. In addition, markers 62A to 62D are provided at four corners of the housing of the panel base portion 58. Note that, among the markers 62A to 62D, the markers 62A and 62B are not illustrated in FIG. 1, and the markers 62B and 62C are not illustrated in FIG. 4 since the markers are in the shadow of the panel base portion 58. The marker 62A to the marker 62D are markers that can be respectively identified, and the identification of the marker 62A to the marker 62D can be performed in at least an optical image captured by the optical camera 40 of the radiation source unit 10. In the present embodiment, the marker 42A and the marker 62A are the same marker, the marker 42B and the marker 62B are the same marker, the marker 42C and the marker 62C are the same marker, and the marker 42D and the marker 62D are the same marker. Note that, in the following, in a case where the marker 62A to the marker 62D are collectively referred to, the marker 62A to the marker 62D are simply referred to as the “marker 62”. The optical camera 60 of the present embodiment is an example of a second detection unit according to the present disclosure.

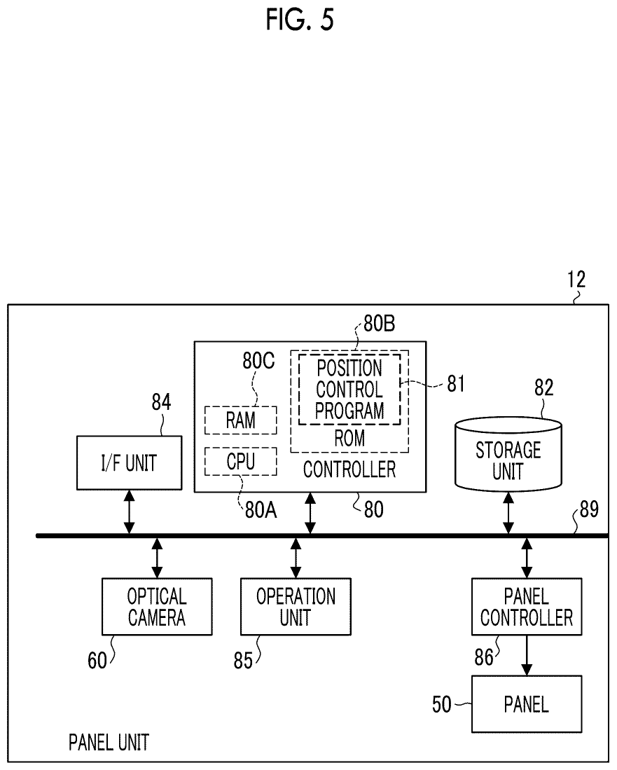

Furthermore, the configuration of the panel unit 12 will be described with reference to FIG. 5. As illustrated in FIG. 5, the panel unit 12 of the present embodiment further comprises a controller 80, a storage unit 82, an operation unit 85, and a panel controller 86. The optical camera 60, the controller 80, the storage unit 82, the operation unit 85, and the panel controller 86 are connected to each other via a bus 89, such as a system bus or a control bus, such that various kinds of information can be exchanged.

The controller 80 includes a CPU 80A, a ROM 80B, and a RAM 80C. The CPU 80A controls the entire panel unit 12. The ROM 80B stores in advance various programs including a position control program 81 to be executed by the CPU 80A. The RAM 80C temporarily stores various kinds of data. Note that the controller 70 and the controller 80 described above are examples of a control device of the present disclosure.

The storage unit 82 stores various kinds of information related to imaging. The storage unit 82 is realized by, for example, a storage medium such as an HDD, an SSD, and a flash memory.

An I/F unit 84 performs communication of various kinds of information with the radiation source unit 10 or an external apparatus by wired communication or wireless communication.

The operation unit 85 is used for a user to input an instruction related to imaging, various kinds of information, and the like. Note that the operation unit 85 is not particularly limited, and examples of the operation unit 85 include various switches, a touch panel, a touch pen, a mouse, and the like.

The panel controller 86 controls the driving of the panel 50 on the basis of the control of the controller 80. The panel 50 detects radiation and outputs a radiation image under the control of the controller 80.

Next, an operation of the radiography system 1 of the present embodiment will be described. The radiography system 1 of the present embodiment controls the positions of the radiation source unit 10 and the panel unit 12 such that the radiation source unit 10 and the panel unit 12 face each other. Here, the operation of controlling the positions of the radiation source unit 10 and the panel unit 12 will be described.

In the radiography system 1 of the present embodiment, an image of the marker 42 included in a first optical image and an image of the marker 62 included in a second optical image are different from each other according to the relative position and the relative posture between the radiation source unit 10 and the panel unit 12. Specifically, among the four types (A to D) of markers, the image of which marker is included in the optical image is different.

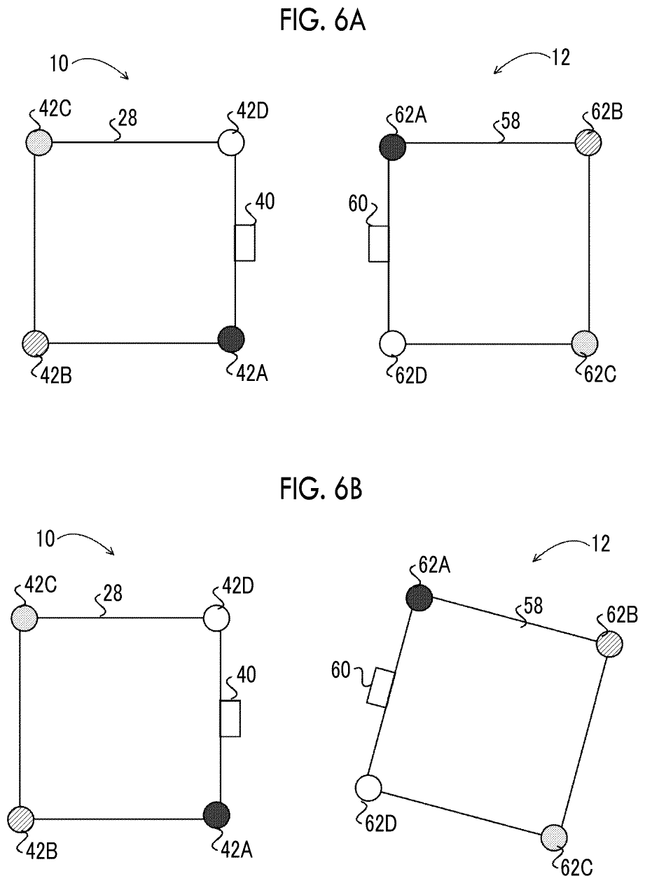

FIG. 6A and FIG. 6B schematically illustrate the radiation source base portion 28, the optical camera 40, the marker 42, the panel base portion 58, the optical camera 60, and the marker 62. FIG. 6A illustrates a case where the radiation source unit 10 and the panel unit 12 face each other. On the other hand, FIG. 6B illustrates a case where the radiation source unit 10 and the panel unit 12 are not facing each other, the relative positions thereof are shifted, and the relative postures thereof are different from each other.

As illustrated in FIG. 6A, in a case where the radiation source unit 10 and the panel unit 12 face each other, the marker 62A and the marker 62D are included in the first optical image obtained by the optical camera 40. In addition, the marker 42A and the marker 42D are included in the second optical image obtained by the optical camera 60. Since the markers 42A to 42D and the markers 62A to 62D are the same and the radiation source base portion 28 and the panel base portion 58 have a common shape, in a case where the radiation source unit 10 and the panel unit 12 face each other, the state of the marker 62 included in the first optical image and the state of the marker 42 included in the second optical image are the same.

On the other hand, as illustrated in FIG. 6B, in a case where the radiation source unit 10 and the panel unit 12 are not facing each other, the marker 62A, the marker 62C, and the marker 62D are included in the first optical image obtained by the optical camera 40. In addition, the marker 42A and the marker 42D are included in the second optical image obtained by the optical camera 60.

In this way, in a case where the radiation source unit 10 and the panel unit 12 are not facing each other, the state of the marker 62 included in the first optical image and the state of the marker 42 included in the second optical image are not the same. In addition, both the first optical image and the second optical image are images in which the states of the markers 42 and 62 are different from a case where the radiation source unit 10 and the panel unit 12 face each other.

Therefore, in the radiography system 1 of the present embodiment, the relative position and the relative posture of the radiation source unit 10 and the panel unit 12 are controlled on the basis of at least one of the first optical image or the second optical image.

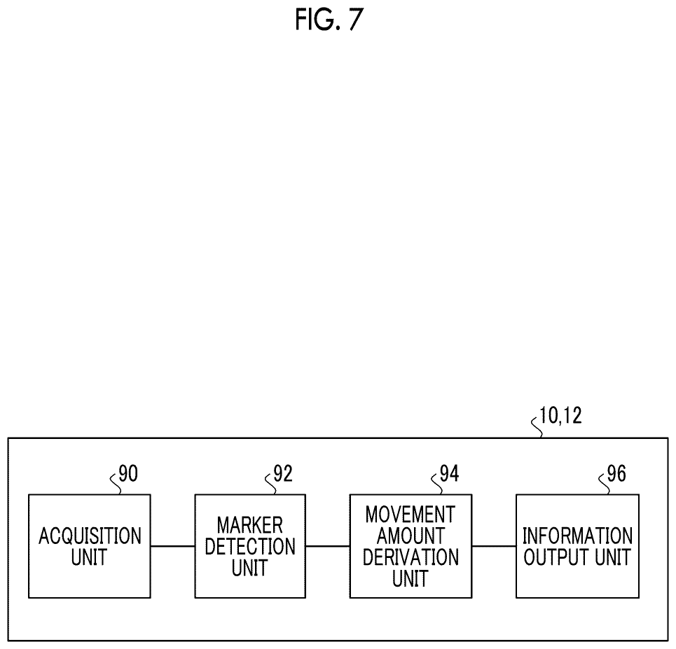

FIG. 7 is a functional block diagram illustrating an example of a functional configuration of the radiation source unit 10 and the panel unit 12. In the present embodiment, as illustrated in FIG. 7, both the radiation source unit 10 and the panel unit 12 comprise an acquisition unit 90, a marker detection unit 92, a movement amount derivation unit 94, and an information output unit 96.

First, functions of each unit in the radiation source unit 10 will be described. In the radiation source unit 10, the CPU 70A of the controller 70 executes the position control program 71 to function as the acquisition unit 90, the marker detection unit 92, the movement amount derivation unit 94, and the information output unit 96. The acquisition unit 90 acquires an optical image (referred to as a “first optical image” in the present embodiment) captured by the optical camera 40. In addition, the acquisition unit 90 acquires at least one of facing information or movement information, which will be described in detail later, from the panel unit 12.

The marker detection unit 92 detects an image of the marker 62 included in the first optical image acquired from the optical camera 40. Note that a method by which the marker detection unit 92 detects an image of the marker 62 from the first optical image is not limited. For example, an image analysis may be performed on the first optical image to detect whether or not an image corresponding to a color or a shape as a feature amount of each marker 62 is present.

The movement amount derivation unit 94 derives a displacement amount of the position of the image of the marker 62 from the position of the image of the marker 62 detected by the marker detection unit 92 and the position of the image of the marker 62 included in the first optical image (hereinafter, referred to as a “first reference optical image”) captured by the optical camera 40 in a case where the radiation source unit 10 and the panel unit 12 face each other. In addition, the movement amount derivation unit 94 derives a movement amount and a movement direction of the panel unit 12 from the derived displacement amount. Note that a method by which the movement amount derivation unit 94 derives the movement amount and the movement direction is not limited, and for example, correspondence relationship information representing a correspondence relationship between the displacement amount of the image of the marker 62 and the movement amount and the movement direction of the panel unit 12 may be obtained in advance, and then the movement amount and the movement direction may be derived on the basis of the correspondence relationship information.

Note that the movement amount derivation unit 94 determines that the radiation source unit 10 and the panel unit 12 face each other in a case where the displacement amount is “0”.

The information output unit 96 outputs a derivation result of the movement amount derivation unit 94. Specifically, in a case where the movement amount derivation unit 94 determines that the radiation source unit 10 and the panel unit 12 face each other, the information output unit 96 of the radiation source unit 10 outputs facing information indicating that the radiation source unit 10 and the panel unit 12 face each other to the display unit 32. On the other hand, in a case where the movement amount derivation unit 94 derives the movement amount and the movement direction, the information output unit 96 of the radiation source unit 10 outputs the movement information indicating the derived movement amount and movement direction to the display unit 32.

Next, functions of each unit in the panel unit 12 will be described. In the panel unit 12, the CPU 80A of the controller 80 executes the position control program 81 to function as the acquisition unit 90, the marker detection unit 92, the movement amount derivation unit 94, and the information output unit 96. The acquisition unit 90 acquires an optical image (referred to as a “second optical image” in the present embodiment) captured by the optical camera 60. In addition, the acquisition unit 90 acquires reference unit information to be described in detail later from the radiation source unit 10.

The marker detection unit 92 detects an image of the marker 42 included in the second optical image acquired from the optical camera 60. Note that a method by which the marker detection unit 92 detects an image of the marker 42 from the second optical image is not limited. For example, an image analysis may be performed on the second optical image to detect whether or not an image corresponding to a color or a shape as a feature amount of each marker 42 is present. Note that, in the markers 42 and the markers 62, the same markers are arranged in the same states in each of the radiation source base portion 28 and the panel base portion 58, and therefore, a method of detecting the image of the marker 62 from the first optical image and a method of detecting the image of the marker 42 from the second optical image can be the same method.

The movement amount derivation unit 94 derives a displacement amount of the position of the image of the marker 42 from the position of the image of the marker 42 detected by the marker detection unit 92 and the position of the image of the marker 42 included in the second optical image (hereinafter, referred to as a “second reference optical image”) captured by the optical camera 60 in a case where the radiation source unit 10 and the panel unit 12 face each other. In addition, the movement amount derivation unit 94 derives a movement amount and a movement direction of the radiation source unit 10 from the derived displacement amount. Note that a method by which the movement amount derivation unit 94 derives the movement amount and the movement direction is not limited, and for example, correspondence relationship information representing a correspondence relationship between the displacement amount of the image of the marker 42 and the movement amount and the movement direction of the radiation source unit 10 may be obtained in advance, and then the movement amount and the movement direction may be derived on the basis of the correspondence relationship information.

Note that the movement amount derivation unit 94 determines that the radiation source unit 10 and the panel unit 12 face each other in a case where the displacement amount is “0”.

The information output unit 96 outputs a derivation result of the movement amount derivation unit 94. Specifically, in a case where the movement amount derivation unit 94 determines that the radiation source unit 10 and the panel unit 12 face each other, the information output unit 96 of the panel unit 12 outputs facing information indicating that the radiation source unit 10 and the panel unit 12 face each other to the radiation source unit 10. On the other hand, in a case where the movement amount derivation unit 94 derives the movement amount and the movement direction, the information output unit 96 of the panel unit 12 outputs the movement information indicating the derived movement amount and movement direction to the radiation source unit 10.

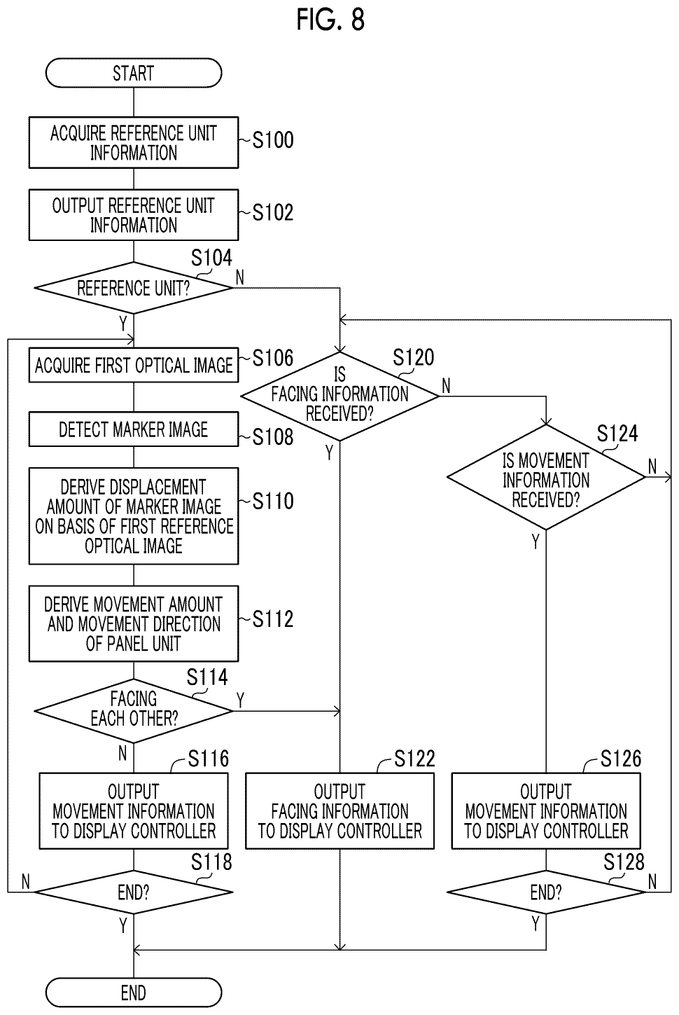

Next, position control processing executed by the radiation source unit 10 will be described. FIG. 8 illustrates a flowchart representing an example of a flow of the position control processing executed by the radiation source unit 10.

In step S100 of FIG. 8, the acquisition unit 90 acquires the reference unit information. In the present embodiment, the position of any one of the radiation source unit 10 or the panel unit 12 is fixed, and the other unit is moved with reference to the position of the fixed unit, whereby the radiation source unit 10 and the panel unit 12 are made to face each other. Therefore, the acquisition unit 90 acquires the reference unit information representing which of the radiation source unit 10 and the panel unit 12 is the reference unit. Note that the reference unit information is input by the user using, for example, the operation unit 30.

In next step S102, the acquisition unit 90 outputs the acquired reference unit information to the panel unit 12.

In next step S104, the acquisition unit 90 determines whether or not the host unit is the reference unit. That is, it is determined whether or not the reference unit is the radiation source unit 10. In a case where the reference unit is the radiation source unit 10, the determination in step S104 is an affirmative determination, and the processing proceeds to step S106.

In step S106, the acquisition unit 90 acquires the first optical image from the optical camera 40.

In next step S108, the marker detection unit 92 detects the image of the marker 62 from the first optical image as described above.

In next step S110, as described above, the movement amount derivation unit 94 derives a displacement amount of the position of the image of the marker 62 included in the first optical image with respect to the position of the image of the marker 62 included in the first reference optical image.

In next step S112, the movement amount derivation unit 94 derives the movement amount and the movement direction of the panel unit 12 on the basis of the derived displacement amount as described above.

In the next step S114, the movement amount derivation unit 94 determines whether or not the radiation source unit 10 and the panel unit 12 face each other. In a case where the units are not facing each other, a negative determination is made in step S114, and the processing proceeds to step S116.

In step S116, the information output unit 96 outputs the movement information indicating the movement amount and the movement direction to the display unit 32 as described above. The user moves the panel unit 12 by referring to the movement information displayed on the display unit 32.

In next step S118, the acquisition unit 90 determines whether or not to end the position control processing illustrated in FIG. 8. In a case where the processing is not to be ended, the determination is a negative determination, the processing returns to step S106, and the processing in steps S106 to S116 is repeated. On the other hand, in a case where an instruction to end the processing is received or the like, the determination in step S118 is an affirmative determination, and the position control processing illustrated in FIG. 8 is ended.

On the other hand, in a case where the radiation source unit 10 and the panel unit 12 face each other, the determination in step S114 is an affirmative determination, and the processing proceeds to step S122.

In step S122, as described above, the information output unit 96 outputs information indicating that the radiation source unit 10 and the panel unit 12 face each other, that is, the information indicating that the radiation source unit 10 and the panel unit 12 are disposed in correct states, to the display unit 32. The user recognizes that the disposition of the radiation source unit 10 and the panel unit 12 is completed by referring to the facing information displayed on the display unit 32. In a case where step S122 is ended, the position control processing illustrated in FIG. 8 is ended.

On the other hand, in a case where the reference unit is the panel unit 12, the determination in step S104 is a negative determination, and the processing proceeds to step S120. In step S120, the acquisition unit 90 determines whether or not the facing information is received from the panel unit 12. In a case where the facing information is received, the determination in step S120 is an affirmative determination, and the processing proceeds to step S122. On the other hand, in a case where the facing information is not received, the determination in step S120 is a negative determination, and the processing proceeds to step S124.

In step S124, the acquisition unit 90 determines whether or not the movement information is received from the panel unit 12. In a case where the movement information is not received, the determination in step S124 is a negative determination, and the processing returns to step S120. On the other hand, in a case where the movement information is received, the determination in step S124 is an affirmative determination, and the processing proceeds to step S126. In step S126, the information output unit 96 outputs the movement information indicating the movement amount and the movement direction to the display unit 32 as described above. The user moves the radiation source unit 10 by referring to the movement information displayed on the display unit 32.

In next step S128, the acquisition unit 90 determines whether or not to end the position control processing illustrated in FIG. 8. In a case where the processing is not to be ended, the determination is a negative determination, the processing returns to step S120, and the processing in steps S120 to S126 is repeated. On the other hand, in a case where an instruction to end the processing is received or the like, the determination in step S128 is an affirmative determination, and the position control processing illustrated in FIG. 8 is ended.

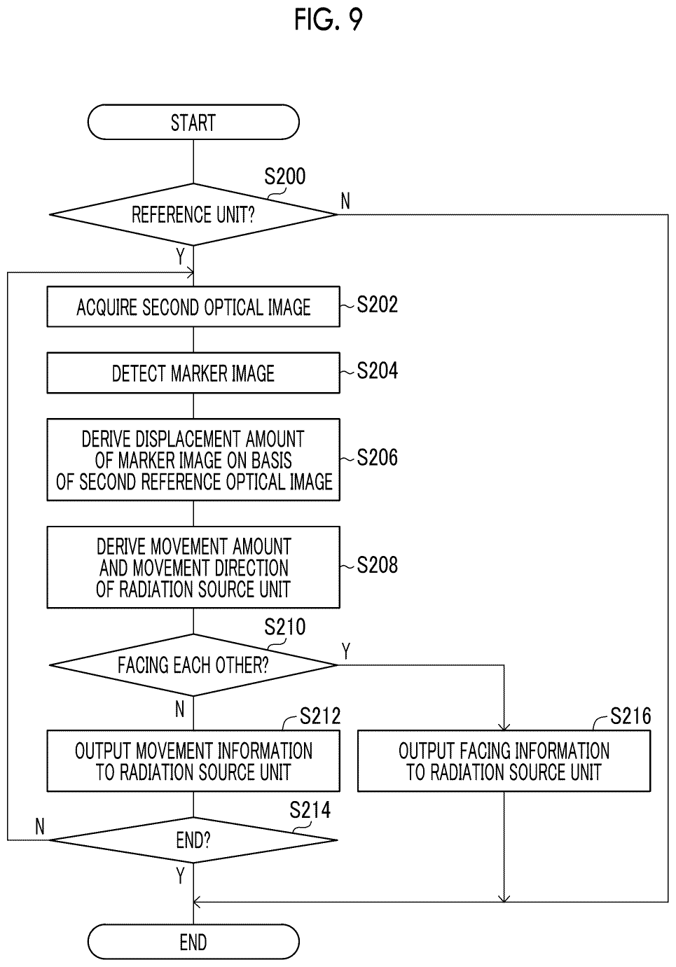

Next, position control processing executed by the panel unit 12 will be described. FIG. 9 illustrates a flowchart representing an example of a flow of the position control processing executed by the panel unit 12. For example, in the present embodiment, the position control processing illustrated in FIG. 9 is executed in a case where the reference unit information is received from the radiation source unit 10.

In step S200 in FIG. 9, the acquisition unit 90 determines whether or not the host unit is the reference unit. That is, it is determined whether or not the reference unit is the panel unit 12.

In a case where the reference unit is not the panel unit 12, the determination is a negative determination, and the position control processing illustrated in FIG. 9 is ended. On the other hand, in a case where the reference unit is the panel unit 12, the determination in step S200 is an affirmative determination, and the processing proceeds to step S202.

In step S202, the acquisition unit 90 acquires the second optical image from the optical camera 60.

In next step S204, the marker detection unit 92 detects the image of the marker 42 from the second optical image as described above.

In next step S206, as described above, the movement amount derivation unit 94 derives a displacement amount of the position of the image of the marker 42 included in the second optical image with respect to the position of the image of the marker 42 included in the second reference optical image.

In next step S208, the movement amount derivation unit 94 derives the movement amount and the movement direction of the radiation source unit 10 on the basis of the derived displacement amount as described above.

In the next step S210, the movement amount derivation unit 94 determines whether or not the radiation source unit 10 and the panel unit 12 face each other. In a case where the units are not facing each other, a negative determination is made in step S210, and the processing proceeds to step S212.

In step S212, the information output unit 96 outputs the movement information indicating the movement amount and the movement direction to the radiation source unit 10 as described above. Accordingly, as described above, the movement information representing the movement direction and the movement amount for moving the radiation source unit 10 is displayed on the display unit 32 of the radiation source unit 10. The user moves the radiation source unit 10 by referring to the movement information displayed on the display unit 32.

In next step S214, the acquisition unit 90 determines whether or not to end the position control processing illustrated in FIG. 9. In a case where the processing is not to be ended, the determination is a negative determination, the processing returns to step S202, and the processing in steps S202 to S212 is repeated. On the other hand, in a case where an instruction to end the processing is received or the like, the determination in step S214 is an affirmative determination, and the position control processing illustrated in FIG. 9 is ended.

On the other hand, in a case where the radiation source unit 10 and the panel unit 12 face each other, the determination in step S210 is an affirmative determination, and the processing proceeds to step S216.

In step S216, as described above, the information output unit 96 outputs information indicating that the radiation source unit 10 and the panel unit 12 face each other, that is, the information indicating that the radiation source unit 10 and the panel unit 12 are disposed in correct states, to the radiation source unit 10. As a result, as described above, the facing information is displayed on the display unit 32 of the radiation source unit 10. Accordingly, the user recognizes that the disposition of the radiation source unit 10 and the panel unit 12 is completed by referring to the facing information displayed on the display unit 32 of the radiation source unit 10. In a case where step S216 is ended, the position control processing illustrated in FIG. 9 is ended.

As described above, in the radiography system 1 according to the embodiment described above, the radiation source base portion 28 of the radiation source unit 10 and the panel base portion 58 of the panel unit 12 have a common shape.

The relative position and the relative posture of the radiation source unit 10 and the panel unit 12 are controlled on the basis of at least one of the optical image captured by the optical camera 60 or the optical image captured by the optical camera 40.

Therefore, with the radiography system 1 according to the embodiment described above, it is possible to easily perform the alignment of the radiation source unit and the panel unit.

In addition, in the embodiment described above, since the radiation source base portion 28 and the panel base portion 58 have a common shape and have the same mechanism (a combination of the optical camera 40 and the marker 62 and a combination of the optical camera 60 and the marker 42) for detecting the relative positions and the relative postures of each other, the robustness can be improved. In addition, the cost related to the radiation source unit 10 and the panel unit 12 can be reduced.

In addition, in the above description, the form has been described in which the user moves the radiation source unit 10 and the panel unit 12, but the radiation source unit 10 and the panel unit 12 may be automatically moved by the control of the controller 70 or the controller 80.

In addition, in the embodiment described above, the form has been described in which the reference unit derives the movement information for moving the other unit, but a form in which the other unit itself derives the movement information for moving the host unit may be adopted.

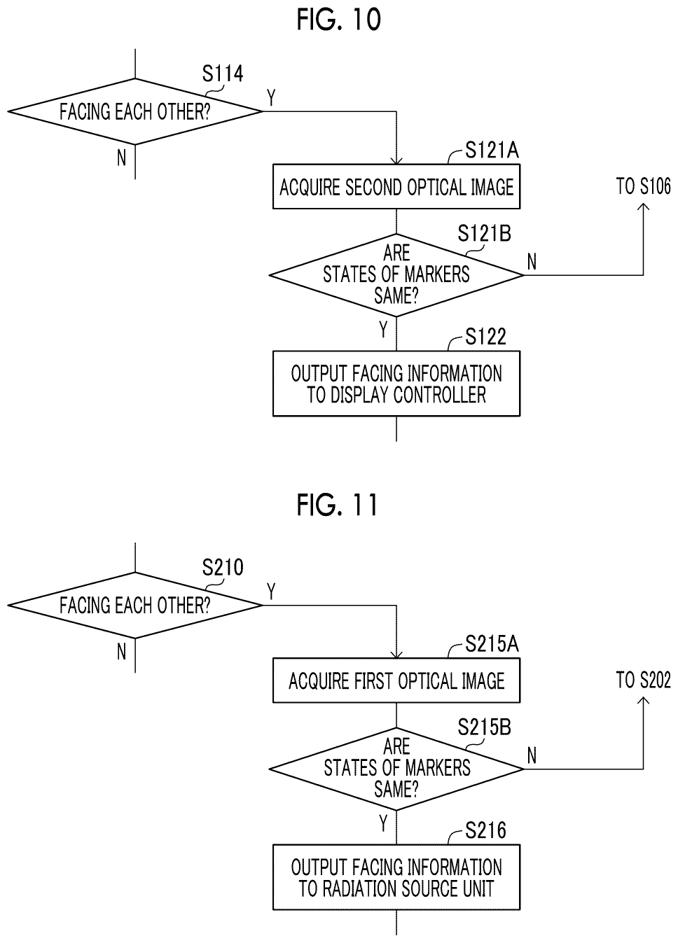

In addition, in the embodiment described above, the form of using only one of the first optical image and the second optical image has been described, but both the first optical image and the second optical image may be used. For example, it may be determined that the radiation source unit 10 and the panel unit 12 face each other on the basis of the first optical image, and it may be determined that the radiation source unit 10 and the panel unit 12 face each other in a case where the state of the image of the marker 62 included in the first optical image and the state of the image of the marker 42 included in the second optical image can be regarded to be the same. In this case, as illustrated in FIG. 10, the processing of steps S121A and S121B is added before the processing of step S122 in the position control processing (refer to FIG. 8) described above. In step S121A in FIG. 10, the acquisition unit 90 acquires the second optical image captured by the optical camera 60 from the panel unit 12. Specifically, an instruction to image the radiation source unit 10 by the optical camera 60 is output to the panel unit 12, and the acquisition unit 90 acquires the second optical image captured in accordance with the instruction. In next step S121B, the movement amount derivation unit 94 determines whether or not the state of the image of the marker 62 included in the first optical image and the state of the image of the marker 42 included in the second optical image can be regarded to be the same. In a case where the states can be regarded to be the same, the determination in step S121B is an affirmative determination, and the processing proceeds to step S122. On the other hand, in a case where the states cannot be regarded to be the same, the determination in step S121B is a negative determination, the processing returns to step S106, and the above-described processing is repeated.

Similarly, it may be determined that the radiation source unit 10 and the panel unit 12 face each other on the basis of the second optical image, and it may be determined that the radiation source unit 10 and the panel unit 12 face each other in a case where the state of the image of the marker 62 included in the first optical image and the state of the image of the marker 42 included in the second optical image can be regarded to be the same. In this case, as illustrated in FIG. 11, the processing of steps S215A and S215B is added before the processing of step S216 in the position control processing (refer to FIG. 9) described above. In step S215A in FIG. 11, the acquisition unit 90 acquires the first optical image captured by the optical camera 40 from the radiation source unit 10. Specifically, an instruction to image the panel unit 12 by the optical camera 40 is output to the radiation source unit 10, and the acquisition unit 90 acquires the first optical image captured in accordance with the instruction. In next step S215B, the movement amount derivation unit 94 determines whether or not the state of the image of the marker 62 included in the first optical image and the state of the image of the marker 42 included in the second optical image can be regarded to be the same. In a case where the state of the image of the marker 42 can be regarded to be the same, the determination in step S215B is an affirmative determination, and the processing proceeds to step S216. On the other hand, in a case where the state of the image of the marker 42 cannot be regarded to be the same, the determination in step S215B is a negative determination, the processing returns to step S202, and the above-described processing is repeated.

As described above, by using both the first optical image and the second optical image, the accuracy of the alignment of the radiation source unit 10 and the panel unit 12 can be further improved.

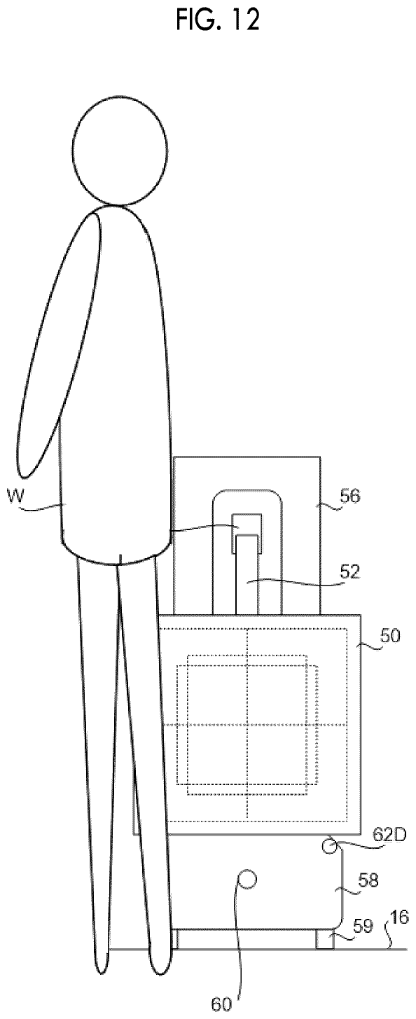

In addition, in the first optical image or the second optical image, the marker 42 or the marker 62 may be hidden by a subject or the like. For example, in the example illustrated in FIG. 12, the marker 62A provided on the panel base portion 58 of the panel unit 12 is hidden by a subject W. In the case illustrated in FIG. 12, even in a state where the radiation source unit 10 and the panel unit 12 face each other, the image of the marker 62A that should originally be included is not included in the first optical image captured by the optical camera 40 of the radiation source unit 10. On the other hand, the images of both the markers 42A and 42D are included in the second optical image captured by the optical camera 60 of the panel unit 12. Therefore, it is preferable to perform the alignment of the radiation source unit 10 and the panel unit 12 by using the second optical image without using the first optical image. In this way, a form may be adopted in which, among the first optical image and the second optical image, an optical image not including an image of a predetermined marker to be imaged in a case where the units are facing each other is not used, and an optical image including an image of a predetermined marker is used to derive the movement amount and the movement direction.

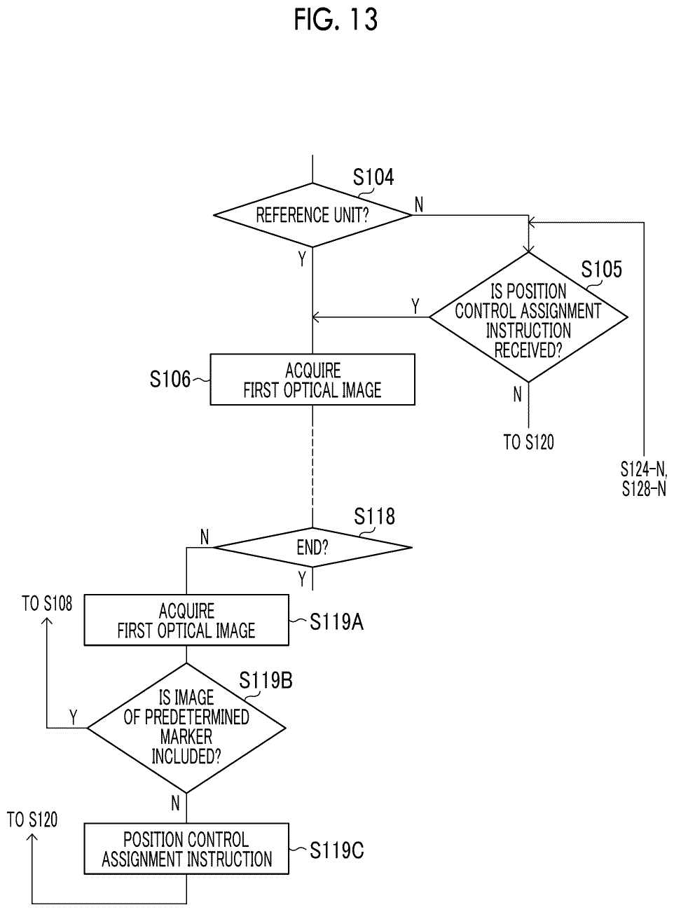

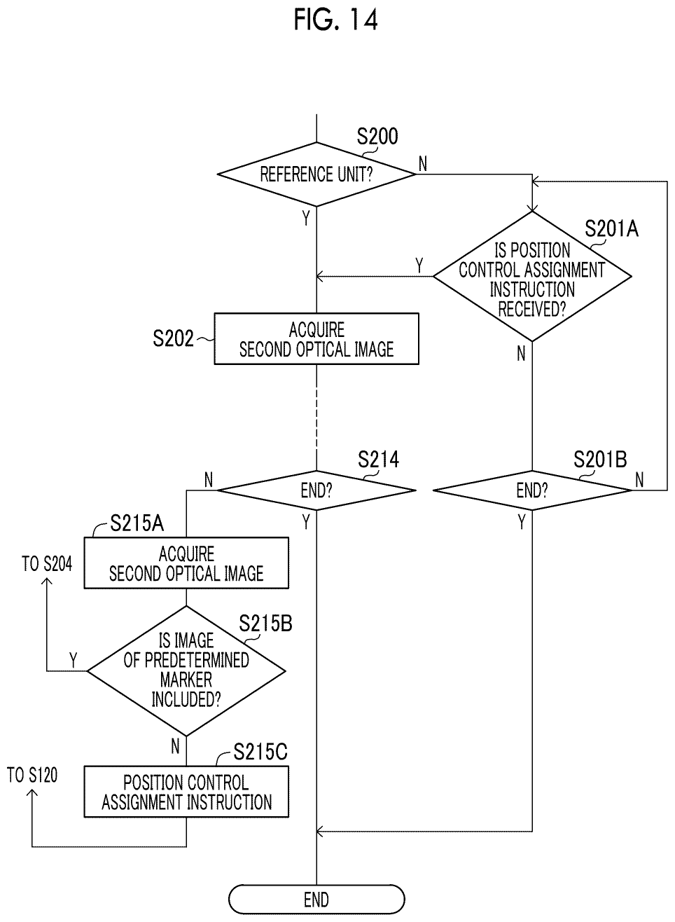

For example, in the position control processing (refer to FIGS. 8 and 9) described above, the subject W may enter between the radiation source unit 10 and the panel unit 12 before the radiation source unit 10 and the panel unit 12 face each other, so that the image of the predetermined marker may not be included in the first optical image or the second optical image. In this case, a form may be adopted in which the movement amount and the movement direction are derived on the basis of the optical image including the image of the predetermined marker among the first optical image and the second optical image. An example of the form will be described with reference to the flowchart representing an example of the flow of the position control processing illustrated in FIGS. 13 and 14.

FIG. 13 illustrates a flowchart representing an example of a flow of the position control processing executed by the radiation source unit 10 of the form. The position control processing illustrated in FIG. 13 is different from the position control processing (refer to FIG. 8) according to the embodiment described above, in the processing that follows a negative determination in step S104. In the present form, as illustrated in FIG. 13, in a case where a negative determination is made in step S104, the processing proceeds to step S105. In addition, as illustrated in FIG. 13, in the present form, in a case where a negative determination is made in step S124 and in a case where a negative determination is made in step S128, the processing proceeds to step S105.

In step S105, the acquisition unit 90 determines whether or not a position control assignment instruction is received from the panel unit 12. The position control assignment instruction is an instruction for assignment of processing for deriving the movement amount and the movement direction for causing the radiation source unit 10 and the panel unit 12 to face each other. In the radiation source unit 10, in a case where the position control assignment instruction is received, the processing of steps S106 to S112 is executed to derive the movement amount and the movement direction of the panel unit 12. Therefore, in a case where the position control assignment instruction is received, the determination in step S105 is an affirmative determination, and the processing proceeds to step S106. On the other hand, in a case where the position control assignment instruction is not received, the determination in step S105 is a negative determination, and the processing proceeds to step S120.

In addition, the position control processing illustrated in FIG. 13 is different from the position control processing (refer to FIG. 8) according to the embodiment described above, in the processing that follows a negative determination in step S118. In the present form, as illustrated in FIG. 13, in a case where a negative determination is made in step S118, the processing proceeds to step S119A.

In step S119A, the acquisition unit 90 acquires the first optical image from the optical camera 40.

In next step S119B, the marker detection unit 92 determines whether or not the image of the predetermined marker is included in the acquired first optical image. As an example, here, it is determined whether or not, as the image of the predetermined marker, the images of the markers 62A and 62D are included in the first optical image. In a case where the image of the predetermined marker is included in the first optical image, since the position control is performed on the basis of the first optical image as it is, the determination in step S119B is an affirmative determination, and the processing proceeds to step S108. On the other hand, in a case where the image of the predetermined marker is not included in the first optical image, the determination in step S119B is a negative determination, and the processing proceeds to step S119C.

In step S119C, the information output unit 96 outputs the position control assignment instruction described above to the panel unit 12, and then the processing proceeds to step S120.

On the other hand, FIG. 14 illustrates a flowchart representing an example of a flow of the position control processing executed by the panel unit 12 of the form.

The position control processing illustrated in FIG. 14 is different from the position control processing (refer to FIG. 9) according to the embodiment described above, in the processing that follows a negative determination in step S200. In the present form, as illustrated in FIG. 14, in a case where a negative determination is made in step S200, the processing proceeds to step S201A.

In step S201A, the acquisition unit 90 determines whether or not the position control assignment instruction is received from the radiation source unit 10. In the panel unit 12, in a case where the position control assignment instruction is received, the processing of steps S202 to S208 is executed to derive the movement amount and the movement direction of the radiation source unit 10. Therefore, in a case where the position control assignment instruction is received, the determination in step S201A is an affirmative determination, and the processing proceeds to step S202. On the other hand, in a case where the position control assignment instruction is not received, the determination in step S201A is a negative determination, and the processing proceeds to step S201B.

In step S201B, the acquisition unit 90 determines whether or not to end the position control processing illustrated in FIG. 13. In a case where the processing is not to be ended, the determination is a negative determination, the processing returns to step S201A. On the other hand, in a case where an instruction to end the processing is received or the like, the determination in step S201B is an affirmative determination, and the position control processing illustrated in FIG. 14 is ended.

In addition, the position control processing illustrated in FIG. 14 is different from the position control processing (refer to FIG. 9) according to the embodiment described above, in the processing that follows a negative determination in step S214. In the present form, as illustrated in FIG. 14, in a case where a negative determination is made in step S214, the processing proceeds to step S215A.

In step S215A, the acquisition unit 90 acquires the second optical image from the optical camera 60.

In next step S215B, the marker detection unit 92 determines whether or not the image of the predetermined marker is included in the acquired second optical image. As an example, here, it is determined whether or not, as the image of the predetermined marker, the images of the markers 42A and 42D are included in the second optical image. In a case where the image of the predetermined marker is included in the second optical image, since the position control is performed on the basis of the second optical image as it is, the determination in step S215B is an affirmative determination, and the processing proceeds to step S204. On the other hand, in a case where the image of the predetermined marker is not included in the second optical image, the determination in step S215B is a negative determination, and the processing proceeds to step S215C.

In step S215C, the information output unit 96 outputs the position control assignment instruction described above to the radiation source unit 10, and then the processing proceeds to step S120.

As described above, in the form illustrated in FIGS. 13 and 14, in a case where the subject W enters or the like and thus the state in which the image of the predetermined marker is not included in the first optical image or the second optical image is made in the middle of the position control processing, the alignment of the radiation source unit 10 and the panel unit 12 is performed using the optical image in which the image of the predetermined marker is included, among the first optical image and the second optical image. Therefore, in the form illustrated in FIGS. 13 and 14, the accuracy of the alignment can be further improved, and the robustness can be improved.

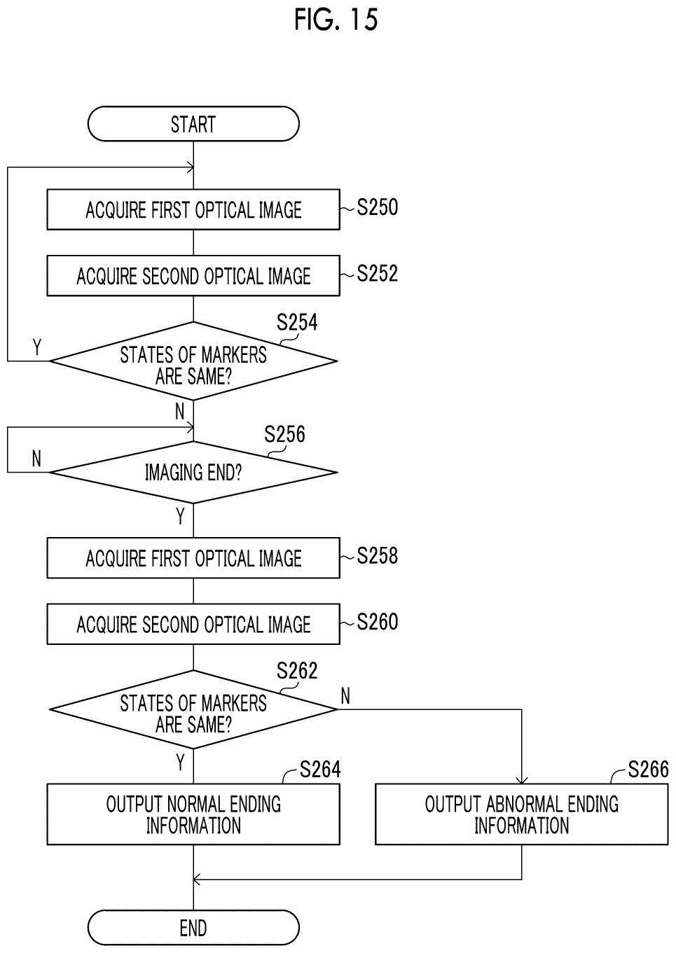

In addition, in addition to the embodiment described above, after the imaging after the radiation, during the imaging of the radiation image, position checking processing of determining whether or not there is misalignment between the radiation source unit 10 and the panel unit 12 may be executed by determining whether or not the radiation source unit 10 and the panel unit 12 are facing each other. The position checking processing will be described with reference to FIG. 15. FIG. 15 illustrates a flowchart representing an example of a flow of the position checking processing executed by the panel unit 12. The position checking processing illustrated in FIG. 15 is executed after the position control processing (refer to FIG. 9) described above is ended.

In step S250 in FIG. 15, the acquisition unit 90 acquires the first optical image captured by the optical camera 40 from the radiation source unit 10. Specifically, an instruction to image the panel unit 12 by the optical camera 40 is output to the radiation source unit 10, and the acquisition unit 90 acquires the first optical image captured in accordance with the instruction.

In next step S252, the acquisition unit 90 acquires the second optical image from the optical camera 60.

In next step S254, the movement amount derivation unit 94 determines whether or not the state of the image of the marker 62 included in the first optical image and the state of the image of the marker 42 included in the second optical image can be regarded to be the same. After the radiation source unit 10 and the panel unit 12 face each other by the position control processing, the subject W is positioned in front of the panel 50 of the panel unit 12. Therefore, the marker 62 may be hidden by the subject W (for example, refer to FIG. 12), or the optical camera 60 may be hidden by written data WD. Therefore, in the position checking processing illustrated in FIG. 15, in a case where the state of the image of the marker 62 included in the first optical image and the state of the image of the marker 42 included in the second optical image are not the same, it is regarded that the subject W is positioned for capturing a radiation image.

Therefore, in a case where the state of the image of the marker 62 included in the first optical image and the state of the image of the marker 42 included in the second optical image can be regarded to be the same, the determination in step S254 is an affirmative determination, the processing returns to step S250, and the processing of steps S250 and S252 is repeated. On the other hand, in a case where the state of the image of the marker 62 included in the first optical image and the state of the image of the marker 42 included in the second optical image cannot be regarded to be the same, the determination in step S254 is a negative determination, and the processing proceeds to step S256. In this case, as described above, the subject W is positioned, and the capturing of the radiation image is performed.

Therefore, in step S256, the acquisition unit 90 determines whether or not the capturing of the radiation image is ended. Note that the method by which the acquisition unit 90 determines whether or not the capturing of the radiation image is ended is not limited. For example, in a case where the radiation image is output from the panel 50, the acquisition unit 90 may determine that the capturing of the radiation image is ended. In addition, for example, in a case where the panel unit 12 receives an instruction to end the imaging, input by the user via the operation unit 30 of the panel unit 12, the acquisition unit 90 may determine that the capturing of the radiation image is ended. Until the capturing of the radiation image is ended, the determination in step S256 is a negative determination. On the other hand, in a case where the capturing of the radiation image is ended, the determination in step S256 is an affirmative determination, and the processing proceeds to step S258.

In step S258, the acquisition unit 90 acquires the first optical image captured by the optical camera 40 from the radiation source unit 10 as in step S250.

In next step S260, the acquisition unit 90 acquires the second optical image from the optical camera 60.

In next step S262, the movement amount derivation unit 94 determines whether or not the state of the image of the marker 62 included in the first optical image and the state of the image of the marker 42 included in the second optical image can be regarded to be the same, as in step S254. In a case where the state of the image of the marker 62 included in the first optical image and the state of the image of the marker 42 included in the second optical image can be regarded to be the same, the determination in step S262 is an affirmative determination, and the processing proceeds to step S264. In this case, it can be regarded that the radiation source unit 10 and the panel unit 12 are facing each other even during the capturing of the radiation image.

Therefore, in step S264, the information output unit 96 outputs normal ending information representing that the capturing of the radiation image is ended in a state where the positions of the radiation source unit 10 and the panel unit 12 face each other, that is, in a normal state. In a case where the processing of step S264 is ended, the position checking processing illustrated in FIG. 15 is ended. Note that an output destination of the normal ending information is not limited. For example, the normal ending information may be output to the radiation source unit 10. In this case, information representing that the imaging is ended while the radiation source unit 10 and the panel unit 12 are in the facing state may be displayed on the display unit 32 of the radiation source unit 10, on the basis of the normal ending information. In addition, for example, the normal ending information may be output to a storage destination of the captured radiation image, and stored in association with the captured radiation image.

On the other hand, in a case where the state of the image of the marker 62 included in the first optical image and the state of the image of the marker 42 included in the second optical image cannot be regarded to be the same, the determination in step S262 is a negative determination, and the processing proceeds to step S266. In this case, it can be regarded that the radiation source unit 10 and the panel unit 12 are not facing each other during the capturing of the radiation image.

Therefore, in step S266, the information output unit 96 outputs abnormal ending information representing that the capturing of the radiation image is ended while the positions of the radiation source unit 10 and the panel unit 12 are not in the facing state, that is, are in an abnormal state. In a case where the processing of step S266 is ended, the position checking processing illustrated in FIG. 15 is ended. Note that an output destination of the abnormal ending information is not limited. For example, the abnormal ending information may be output to the radiation source unit 10. In this case, information representing that the imaging is ended in a state where the radiation source unit 10 and the panel unit 12 are not in the facing state may be displayed on the display unit 32 of the radiation source unit 10, on the basis of the abnormal ending information. In addition, for example, the abnormal ending information may be output to the storage destination of the captured radiation image, and stored in association with the captured radiation image.

As described above, with the position checking processing illustrated in FIG. 15, it is possible to authenticate whether or not the radiation source unit 10 and the panel unit 12 are in the facing state even during the capturing of the radiation image. In the radiography system 1, in a case where it is determined that the radiation source unit 10 and the panel unit 12 are not in the facing state, the user can be notified of the determination. For example, the user can determine whether or not re-capturing of the radiation image is to be performed on the basis of the notified abnormal ending information.

Therefore, by performing the position checking processing illustrated in FIG. 15, the user can determine whether or not the positions of the radiation source unit 10 and the panel unit 12 are normal during the capturing of the radiation image. Therefore, the user can perform, for example, re-imaging of the radiation as necessary.

In addition, in the embodiment described above, the form of controlling the relative position and the relative posture of the radiation source unit 10 and the panel unit 12 has been described, but a form of controlling at least one of the relative position or the relative posture may be used.

In addition, in the embodiment described above, the form in which the marker 42 and the marker 62 are used has been described, but the present disclosure is not limited as long as a feature point of each of the radiation source base portion 28 and the panel base portion 58 can be detected by each of the first detection unit and the second detection unit. For example, instead of the marker, a beacon that allows each signal to be identified by differentiating the types of signals may be used. In addition, a form of using a distance measurement device or a distance measurement camera that can detect a distance to a feature point instead of the optical camera or in addition to the optical camera may be adopted. In addition, for example, a form of using a magnetic sensor and the like may be used.

In the embodiments and each modification example described above, for example, as hardware structures of processing units that execute various kinds of processing, such as the acquisition unit 90, the marker detection unit 92, the movement amount derivation unit 94, and the information output unit 96, various processors described below can be used. The various processors include, for example, a programmable logic device (PLD) that is a processor of which the circuit configuration can be changed after manufacture, such as a field-programmable gate array (FPGA), and a dedicated electric circuit that is a processor having a dedicated circuit configuration designed to execute specific processing, such as an application specific integrated circuit (ASIC), in addition to the CPU that is a general-purpose processor which executes software (program) to function as various processing units as described above.

One processing unit may be configured by one of the various processors or a combination of the same or different kinds of two or more processors (for example, a combination of a plurality of FPGAs or a combination of a CPU and an FPGA). In addition, a plurality of processing units may be configured into a single processor.

As an example where a plurality of processing units are configured into one processor, first, there is a form where one processor is configured by a combination of one or more CPUs and software as typified by a computer, such as a client or a server, and this processor functions as a plurality of processing units. Second, as typified by a system-on-chip (SoC) or the like, a form of using a processor for realizing the function of the entire system including a plurality of processing units with one integrated circuit (IC) chip can be exemplified. In this manner, various processing units are configured by using one or more of the above-described various processors as hardware structures.

In addition, specifically, an electric circuit (circuitry) obtained by combining circuit elements, such as semiconductor elements, can be used as the hardware structure of the various processors.

In addition, in the embodiment described above, the aspect has been described in which the position control program 71 is stored (installed) in the ROM 70B in advance and the position control program 81 is stored in the ROM 80B in advance, but the present disclosure is not limited thereto. Each of the position control program 71 and the position control program 81 may be provided in a form of being recorded in a recording medium such as a compact disc read only memory (CD-ROM), a digital versatile disc read only memory (DVD-ROM), and a universal serial bus (USB) memory. In addition, each of the position control program 71 and the position control program 81 may be provided in a form of being downloaded from an external device via a network.

In addition, the configurations and operations of the radiography system 1, the radiation source unit 10, the panel unit 12, and the like described in each embodiment described above are illustrative and may be changed according to a situation, without departing from the scope of the present invention. In addition, it is needless to say that the embodiments and the modification example described above may be appropriately combined.

Regarding the embodiments described above, the following supplementary notes are further disclosed.

### Supplementary Note 1

A radiography system comprising:
a radiation source unit;
a panel unit; and
at least one processor,
wherein the radiation source unit includes
   a radiation emitting unit that includes a radiation source,
   a radiation source base portion that supports the radiation emitting unit and includes a radiation source traveling mechanism for performing a traveling movement, and
   a first detection unit that is provided in the radiation source base portion, the panel unit includes
   a panel that detects radiation emitted from the radiation emitting unit,
   a panel base portion that supports the panel and includes a panel traveling mechanism for performing a traveling movement, and
   a second detection unit that is provided in the panel base portion,
the radiation source base portion and the panel base portion have a common shape, and
the processor further includes a control device that controls at least one of a relative position or a relative posture of the radiation source unit and the panel unit on the basis of at least one of a detection result of the first detection unit or a detection result of the second detection unit.

### Supplementary Note 2

The radiography system according to Supplementary Note 1,
wherein the first detection unit and the second detection unit include an optical camera,
the detection result of the first detection unit is a first optical image captured by the optical camera of the first detection unit, and
the detection result of the second detection unit is a second optical image captured by the optical camera of the second detection unit.

### Supplementary Note 3

The radiography system according to Supplementary Note 1 or 2,
wherein each of a housing of the radiation source base portion and a housing of the panel base portion is provided with a marker, and
the control device controls at least one of the relative position or the relative posture of the radiation source unit and the panel unit on the basis of at least one of an image of the marker provided on the panel base portion included in the first optical image or an image of the marker provided on the radiation source base portion included in the second optical image.

### Supplementary Note 4

The radiography system according to Supplementary Note 3,
wherein in a case where it is determined that a state of the image of the marker provided on the panel base portion included in the first optical image and a state of the image of the marker provided on the radiation source base portion included in the second optical image are the same, the control device determines that the radiation source unit and the panel unit are facing each other.

### Supplementary Note 5

The radiography system according to Supplementary Note 2,
wherein each of a housing of the radiation source base portion and a housing of the panel base portion is provided with a plurality of markers including a predetermined marker, and
the control device controls at least one of the relative position or the relative posture of the radiation source unit and the panel unit on the basis of an optical image in which an image of the predetermined marker is included, among the first optical image and the second optical image.

### Supplementary Note 6

The radiography system according to any one of Supplementary Notes 1 to 5,
wherein the control device derives a movement amount and a movement direction of at least one of the radiation source unit or the panel unit for making positions of the radiation source unit and the panel unit into at least one of a relative position or a relative posture determined in advance, on the basis of the detection result of at least one of the first detection unit or the second detection unit.

### Supplementary Note 7

The radiography system according to Supplementary Note 6,
wherein in a case where the movement amount and the movement direction of the radiation source unit are derived, the control device causes the radiation source unit to perform a traveling movement using the radiation source traveling mechanism, on the basis of the derived movement amount and movement direction, and
in a case where the movement amount and the movement direction of the panel unit are derived, the control device causes the panel unit to perform a traveling movement using the panel traveling mechanism, on the basis of the derived movement amount and movement direction.

### Supplementary Note 8

The radiography system according to Supplementary Note 6,
wherein the control device displays the derived movement amount and movement direction on a display unit.

### Supplementary Note 9

A control method of a radiography system including a radiation source unit and a panel unit, in which the radiation source unit includes a radiation emitting unit that includes a radiation source, a radiation source base portion that supports the radiation emitting unit and includes a radiation source traveling mechanism for performing a traveling movement, and a first detection unit that is provided in the radiation source base portion, the panel unit includes a panel that detects radiation emitted from the radiation emitting unit, a panel base portion that supports the panel and includes a panel traveling mechanism for performing a traveling movement, and a second detection unit that is provided in the panel base portion, and the radiation source base portion and the panel base portion have a common shape, the control method comprising:
via a processor,
controlling at least one of a relative position or a relative posture of the radiation source unit and the panel unit on the basis of at least one of a detection result of the first detection unit or a detection result of the second detection unit.

### Supplementary Note 10

A control program of causing a processor of a control device that controls a radiography system including a radiation source unit and a panel unit, in which the radiation source unit includes a radiation emitting unit that includes a radiation source, a radiation source base portion that supports the radiation emitting unit and includes a radiation source traveling mechanism for performing a traveling movement, and a first detection unit that is provided in the radiation source base portion, the panel unit includes a panel that detects radiation emitted from the radiation emitting unit, a panel base portion that supports the panel and includes a panel traveling mechanism for performing a traveling movement, and a second detection unit that is provided in the panel base portion, and the radiation source base portion and the panel base portion have a common shape, to execute processing of
controlling at least one of a relative position or a relative posture of the radiation source unit and the panel unit on the basis of at least one of a detection result of the first detection unit or a detection result of the second detection unit.

### EXPLANATION OF REFERENCES

1: radiography system
10: radiation source unit
12: panel unit
16: floor surface
20: radiation emitting unit
21: radiation source
22: arm
24: holding part
26: support portion
27, 57: gripping portion
28: radiation source base portion
29, 59: wheel
30, 85: operation unit
32: display unit
40, 60: optical camera
42A to 42D, 62A to 62F: marker
50: panel
52: arm
54: holding part
56: support portion
70, 80: controller70A, 80A: CPU70B, 80B: ROM70C, 80C: RAM
71, 81: position control program
72, 82: storage unit
74, 84: I/F unit
76: radiation source controller
77: display controller
79, 89: bus
90: acquisition unit
92: marker detection unit
94: movement amount derivation unit
96: information output unit
W: subject

What is claimed is:

1. A radiography system comprising:
a radiation source unit;
a panel unit; and
at least one processor,
wherein the radiation source unit includes,
a radiation emitting unit that includes a radiation source,
a radiation source base portion that supports the radiation emitting unit and includes a radiation source traveling mechanism for performing a traveling movement, and
a first detection unit that is provided in the radiation source base portion,
wherein the panel unit includes,
a panel that detects radiation emitted from the radiation emitting unit,
a panel base portion that supports the panel and includes a panel traveling mechanism for performing a traveling movement, and
a second detection unit that is provided in the panel base portion,
wherein the radiation source base portion and the panel base portion have a common shape, and
wherein the processor is configured to control at least one of a relative position or a relative posture of the radiation source unit and the panel unit on the basis of at least one of a detection result of the first detection unit or a detection result of the second detection unit.

2. The radiography system according to claim 1, wherein:
the first detection unit and the second detection unit include an optical camera,
the detection result of the first detection unit is a first optical image captured by the optical camera of the first detection unit, and
the detection result of the second detection unit is a second optical image captured by the optical camera of the second detection unit.

3. The radiography system according to claim 2, wherein:
each of a housing of the radiation source base portion and a housing of the panel base portion is provided with a marker, and
the control device controls at least one of the relative position or the relative posture of the radiation source unit and the panel unit on the basis of at least one of an image of the marker provided on the panel base portion included in the first optical image or an image of the marker provided on the radiation source base portion included in the second optical image.

4. The radiography system according to claim 3, wherein in a case where it is determined that a state of the image of the marker provided on the panel base portion included in the first optical image and a state of the image of the marker provided on the radiation source base portion included in the second optical image are the same, the control device determines that the radiation source unit and the panel unit are facing each other.

5. The radiography system according to claim 2, wherein:
each of a housing of the radiation source base portion and a housing of the panel base portion is provided with a plurality of markers including a predetermined marker, and
the control device controls at least one of the relative position or the relative posture of the radiation source unit and the panel unit on the basis of an optical image in which an image of the predetermined marker is included, among the first optical image and the second optical image.

6. The radiography system according to claim 1, wherein the control device derives a movement amount and a movement direction of at least one of the radiation source unit or the panel unit for making positions of the radiation source unit and the panel unit into at least one of a relative position or a relative posture determined in advance, on the basis of the detection result of at least one of the first detection unit or the second detection unit.

7. The radiography system according to claim 6, wherein:
in a case where the movement amount and the movement direction of the radiation source unit are derived, the control device causes the radiation source unit to perform a traveling movement using the radiation source traveling mechanism, on the basis of the derived movement amount and movement direction, and
in a case where the movement amount and the movement direction of the panel unit are derived, the control device causes the panel unit to perform a traveling movement using the panel traveling mechanism, on the basis of the derived movement amount and movement direction.

8. The radiography system according to claim 6, wherein the control device displays the derived movement amount and movement direction on a display unit.

9. A control method of a radiography system including a radiation source unit and a panel unit, in which the radiation source unit includes a radiation emitting unit that includes a radiation source, a radiation source base portion that supports the radiation emitting unit and includes a radiation source traveling mechanism for performing a traveling movement, and a first detection unit that is provided in the radiation source base portion, the panel unit includes a panel that detects radiation emitted from the radiation emitting unit, a panel base portion that supports the panel and includes a panel traveling mechanism for performing a traveling movement, and a second detection unit that is provided in the panel base portion, and the radiation source base portion and the panel base portion have a common shape, the control method comprising:
via a processor,
controlling at least one of a relative position or a relative posture of the radiation source unit and the panel unit on the basis of at least one of a detection result of the first detection unit or a detection result of the second detection unit.

10. A non-transitory computer readable medium storing a control program for causing a processor of a control device that controls a radiography system including a radiation source unit and a panel unit, in which the radiation source unit includes a radiation emitting unit that includes a radiation source, a radiation source base portion that supports the radiation emitting unit and includes a radiation source traveling mechanism for performing a traveling movement, and a first detection unit that is provided in the radiation source base portion, the panel unit includes a panel that detects radiation emitted from the radiation emitting unit, a panel base portion that supports the panel and includes a panel traveling mechanism for performing a traveling movement, and a second detection unit that is provided in the panel base portion, and the radiation source base portion and the panel base portion have a common shape, to execute processing of
controlling at least one of a relative position or a relative posture of the radiation source unit and the panel unit on the basis of at least one of a detection result of the first detection unit or a detection result of the second detection unit.
